(19) United States
(12) Patent Application Publication
(10) Pub. No.: US 2003/0091560 A1
(43) Pub. Date: May 15, 2003

(54) Composition for Pharmaceutical and Cosmetic Products

(76) Inventor: Hartmut Kobilke, Damsdorf (DE)

(21) Appl. No.: 10/248,271

(22) Filed: Jan. 3, 2003

Related U.S. Application Data
(63) Continuation of application No. PCT/DE01/02589, filed on Jul. 5, 2001.

(30) Foreign Application Priority Data
Jul. 5, 2000 (DE) 100 33 939.5
Sep. 2, 2000 (DE) 200 15 177.0

(51) Int. Cl. A61K 39/395; A61K 31/65; A61K 35/80

(52) U.S. Cl. 424/130.1; 424/195.17; 514/152

(57) ABSTRACT

### Abstract of Disclosure

A composition for pharmaceutical products and cosmetic products contains IgY egg yolk antibodies of SPF hens and microalgae extracts from Spirulina species, Spirulina varieties, and Chlorella species. The composition can also contain oxytetracycline and preservatives or antioxidants. The composition prepared as a salve is used for healing infected wounds and for preventing the infection of wounds.

### Cross Reference to Related Applications

This is a continuation of International Application PCT/DE01/02589 with an international filing date of July 5, 2001, not published in English under PCT Article 21(2), and now abandoned.

### Background of Invention

1. Field of the Invention.

The invention relates to a pharmaceutical composition containing immunoglobulin for the treatment of acute as well as antibiotic-resistant chronic inflammations.

2. Description of the Related Art.

In the following, the term pharmaceutical product is to be understood as encompassing prescription drugs as well as non-prescription drugs, homeopathic medicaments as well as dietary supplements.

A large number of different compositions with different specific effects are known. They include compositions with skin-protecting, skin-building and skin-regenerating, inflammation-inhibiting as well as antimicrobial actions which act neutralizing by way of antibiotic or generally immunological action on wound germs and which contain as active ingredients natural plant-based extracts and/or immunoglobulins or chemotherapeutical agents, vitamins and/or antibiotics.

In general, four groups of compositions and their combinations with one another are known:

- Compositions based purely on plant extracts which are used, in particular, against bacteria and for improving healing of wounds, for cell nutrition in the healing process as well as in the form of inflammation-inhibiting substances. For example, DE 196 34 959 A1 describes a wound-treatment and healing salve which contains calendula oil and further plant-derived components. In WO 97/42963 A2 a so-called "green salve" for the treatment of burns and wounds is described which is comprised substantially of plant-based components, inter alia, calendula, and which has a salve base made of olive oil with added beeswax. The effects of individual plant components are described and the microbial strains affected by them are identified.

- Compositions of a chemotherapeutic (antibiotic) basis, in particular, against certain wound infection germs of poly-causal nature, are described, inter alia, in JP 080 533 68 A (containing the component tetracycline) as well as in WO 88/07364 A1 (containing the component oxytetracycline). Other salves with components of linomycin and chloramphenicol are known.

- Moreover, several salves with simple actions are known, for example, those used against bacteria by way of containing tetracyclines. Such salves are described, inter alia, in the publications WO 88/07364 A1 and JP 08053368 A, and their action is based substantially on the active ingredient of calendula flower extracts and/or tetracycline.

- Compositions based on the general and systemic (inner) actions of serum immunoglobulins of the types IgA, IgM and IgG are known. In the publication WO 94/15640 A1 the use of immunoglobulins IgA, IgM, and IgG isolated from blood serum is proposed as a possibility for specific anti-inflammatory action in the systemic sense (for example, sepsis), less with regard to local application.

The utilization of egg yoke antibodies (IgY) is described in several publications such as WO 95/02612 A1; EP 0 696 596 A1; US 5,367,054; JP 092 392 03 A; DE 196 50 991 A1. However, these publications are concerned exclusively with methods and possibilities of use in regard to passive immunotherapy and diagnostics of humans and animals. WO 98/14209 A1 describes the anti-infectious action of IgY but the disclosure of this publication is limited to oral administration.

An oral composition which contains IgA and immunoglobulin G (IgG) is described in U.S. patent 4,335,099 for the treatment of intestinal infections. In addition, preparations containing 73 % IgA and 26 % IgG, based on the total immunoglobulin contents, are able to reduce the occurrence of necrotic enterocolitis when administered prophylactically to infants having a low birth weight. It is assumed that this effect is based on the formation of an antigen-antibody complex as a result of a high antibody titer against a plurality of potential pathogens and toxins. Such pathogens comprise bacteria, fungi, as well as viruses. It has been described, inter alia, in EP 0 506 651 that IgA, IgG and transferrin act synergistically against bacteria growth. The proportions of the active ingredients are between 0.40 and 0.80 parts by weight of IgG and 0.15 to 0.45 parts by weight transferrin per part by weight IgA.

It was demonstrated that IgA, IgG, and IgM act synergistically with other pharmacologically active compounds such as antibiotics. These immunoglobulins bond supposedly on infectious microorganisms which leads to their agglutination, neutralization or induction of phagocytosis (EP 0 168 830).

Administration of the known salves on humans as well as animals or pets is possible only in rooms at room temperature. Therefore, all known salves and emulsions have the disadvantage that their application at freezing ambient temperatures is not possible or that their effect is incomplete or does not occur at all because penetration of the valuable active ingredients into the wound or inflammation center is not possible.

The prior art thus discloses essentially compositions of monovalant or mono-specific action (antibiotics, anti-inflammatory plant-derived active ingredients or immunoglobulins of the blood serum).

Moreover, Carlander David et al. describe, inter alia, in "Peroral immunotherapy with yolk antibodies for the prevention and treatment of enteric infections", Immunologic Research, Vol. 21, No. 1, February 2000, pp. 1-6, a peroral immunotherapy for the prevention or therapy of bacterial and viral infections in the mouth and gastrointestinal tract by employing IgY which is produced from conventional hens. Also, Hatta et al. describe, inter alia, in "Passive immunization against dental plaque formation in humans: Effect of a mouth rinse containing egg yolk antibodies (IgY) specific to Streptococcus mutans", Caries Research, S. Karger AG, Basel, Switzerland, Vol. 31, No. 4, July 1, 1997, pp. 268-274, a peroral passive immunization with IgY and malva extract against S. mutans for prevention of plaque and carries.

Sugita-Konishi Yoshiko et al. describe in "Immune functions of immunoglobulin Y isolated from egg yolk of hens immunized with various infectious bacteria", Bioscience Biotechnology and Biochemistry, Vol. 60, No. 5, 1996, pp. 886-888, experiments with IgY (polyvalent) - which has been isolated by immunization of conventional hens with a mixture of 26 types of bacteria - and the corresponding results. This concerns the prevention of diseases.

According to DE 299 04 221 U1, IgY is used as a feed additive in animal breeding for prevention of diseases. Mentioned is also a peroral passive immunization with IgY. WO 97/37636 A1 describes the use of IgY, produced by immunization of conventional hens, as an additive to foodstuffs and stimulants for peroral immunization for the prevention of diseases.

An artificial saliva substitute which primarily contains components of saliva of the congenital immune system and contains alternatively also IgY isolated from conventional hens, is described in WO 99/04804 A. Also mentioned are plant-derived extracts in combination with IgY.

Database WPI, Derwent Publications Ltd., London, Great Britain, AN 92-085848 XP 002188430 "Skin cosmetic with prolonged effect - contains (modified) antibody and unusual carrier" describes a cosmetic product which contains functional anti-keratin antibodies.

Wound dressings which contain polysaccharide extracts from algae and lipid extracts of algae are described in Database WPI, Derwent Publications Ltd., London, Great Britain; AN 96-157000 XP 002188432 "Skin adhesive sheet skin injuries treat preparation dry gel shall contain polysaccharide extract sea algae" and in Database WPI , Derwent Publications Ltd., London, Great Britain ; AN 81-94328D XP 002188431 "Lipid prodn. from green algae - includes extn. with alcohol-chloroform mixture at low temp., for use in cosmetics and hygiene products".

French patent 5.279 M and French patent application 2 747 922 A describe a wound salve which contains extracts of Spirulina maxima and a cosmetic composition using natural algae extracts of the type Chlorella.

### Summary of Invention

It is an object of the present invention to provide a pharmaceutical composition for preparing pharmaceutical products and cosmetic products in the form of a special, but still universally employable, composition for the treatment of acute and resistant chronic inflammation reactions of the skin and of the cutaneous mucous membrane of humans and animals. A further object of the invention is to provide combinations of pharmaceutical compositions with which humans and animals can be treated more successfully and which can be used prophylactically, therapeutically, and metaphylactically in humans and animals against wound infections very quickly and in a way that they deeply penetrate into inflammation centers.

The composition should be usable in liquid, paste-like or solid form in very different pharmaceutical products (for example, lotions, wound sprays, salves or powders) as well as in dietary supplements (for example, as juice, tablet, or capsule).

The invention is based on the recognition of mono-specific effects of individual components as well as their combinatory-synergistic effects, in particular, on wounds that heal slowly and are infected, under all weather conditions. The polyvalent composition and its complex synergistic healing effect in regard to antiphlogistic, antimicrobial, and skin-tissue regenerating action is based on the inventive combination of:

-Mono or polyvalent, polyclonal, specific egg yolk antibodies of SPF (specific pathogen free) hens and/or hens raised under semi-SPF conditions (status defined) and kept under proper environmental conditions for the hens and fed with a defined feed for breeding hens. The starting materials important for the antibodies, i.e., the egg yolks, as well as the entire egg, are residue-free and foreign contaminant-free and can be certified. The entire production stretch (chain), starting with the donor hen stock, immunizations, and processing, is accompanied by the certifications "EU Öko Audit" (Ecological Certification by European Community), EU-ISO9001, and GLP (Good Laboratory Practice) certificate. The antibodies contain specific effects against the relevant wound infection germs (bacteria) and against those bacteria that are antibiotic-resistant and disinfectant-resistant (hospitalism problem).

- Calendula flower extract.

- Microalgae extract, preferably in the form of microalgae oils, in the variations aqueous, ethanol-based and CO₂ extracts of Spirulina platensis.

- Antibiotics, preferably oxytetracycline.

The inventive composition is characterized by the important active ingredients IgY egg yolk (monovalent or polyvalent) derived from SPF hens, microalgae extracts, preferably of Spirulina species, and Chlorella species, and calendula flower extract. It can comprise also oxytetracycline as well as α-tocopherol as an antioxidant and ascorbic acid as a natural preservative. In the case of pharmaceutical products in the form of salves, they contain also paraffin oil, lanolin preparation (paraffin oil 15.0 parts; water 20.0 parts; wool fat ad 100 parts), calendula flower extract (fluid extract of calendula 1:2/35 %), oxytetracycline or, as an alternative, without oxytetracycline, as well as α-tocopherol as an antioxidant and ascorbic acid as a natural preservative.

This composition is provided for humans and animals for wound treatment, even for resistant suppurate, inflamed, abscessed, gangrenous-necrotic skin wounds and mucous membrane wounds, infected gangrene in humans, for the therapeutic treatment in the case of burns, chemical burns, scald injuries, frostbite of the skin and of the externally accessible mucous membranes of the ear, the oral cavity, genital and anal areas, for the treatment of furuncles and discharging skin eczema, decubitus, and for the treatment of wound infections caused by multi-resistant bacteria (germs) as well as bacterial wound infections.

The advantageous features of the invention will be explained in more detail based on the following examples wherein the individual features, taken alone or in combination, are considered part of the invention. The invention will be explained in more detail based on a composition of a healing and wound-treatment salve.

### Detailed Description

In the following, the employed active ingredients and components as well as the composition and characteristics will be explained.

1. Salve base.

An important component of the pharmaceutical composition according to the invention in the form of a healing and wound-treatment salve is the selected composition of the salve base comprised of lanolin and paraffin oil which has the effect that the salve applied topically to the skin and/or mucous membranes becomes essentially immediately liquid and, in this way, the specific active ingredients contained therein can penetrate deeply into the wound tissue or into the infected tissue.

- Lanolin is a neutral, well-tolerated, homogenous and paste-like salve base which is produced from 65 percent wool fat (cera lanae), 20 percent water, and 15 percent paraffin oil.

- Paraffin oil (viscous paraffin) is additionally admixed to the salve in an amount of 25 to 30 percent as a clear, colorless and odorless oily liquid of saturated and purified hydrocarbons with the goal that the salve is liquefied immediately by the body heat upon application onto the skin or cutaneous mucous membrane. This property effects the deep penetration into the wound and thus also the excellent depth action of the active ingredients of the salve, in particular, of the antibiotic, antimicrobial-immunizing and nutritive-regeneratively active components.

2. Egg yolk antibody (IgY).

The specific egg yolk antibodies with monovalant or polyvalent specific action and composition were isolated from egg yolks or egg yolk extracts of immunized SPF hens or hens raised under semi-SPF conditions, targeting Staphylococcus aureus, Citrobacter, Entereobacteriacege, Micrococcus, and Streptococcus, Coryne bacteria/Coryneforme (including Actionomyces), Coliforme (including Klebsiella), Neisseria, multi-resistance Staphylococcus as well as other current and relevant germ types in infected wounds or the environment.

These specific polyclonal antibodies react immunologically locally in the wound tissue and on the wound surface to wound antigens in the sense of an antigen-antibody reaction. They neutralize the germs infecting the wound specifically, enhance the macrophage activity, and prevent further germ multiplication and its tissue-destructive effect. The specific egg yolk antibodies have been added in amounts of 5 to 6 percent to the salve batches.

3. Calendula flower extract.

Calendula flower extract (fluid extract of calendula 1:2, 35 %) in liquid preparation as ethanol-based or aqueous extract serves as an antiphlogistic agent, i.e., has excellent anti-inflammatory action on wounds and tissue and is admixed to the salve in quantities of between 3 % to 5 %. The essential active ingredients are triterpene saponines and triterpene alcohols, carotinoides, essential oils and polysaccharides. Allergy-causing side effects have not been observed in patients.

4. Microalgae extracts.

Microalgae extracts, for example, of Spirulina platensis, are added in quantities of 2 to 4 percent to the inventive composition in the form of aqueous, alcohol-based (ethanol), or CO₂ extracts. As a result of the plurality of high-quality ingredients such as:

- High-protein contents with the complete spectrum of essential and non-essential amino acids;

- High-molecular and complex polysaccharides;

- Fats and fatty acids, in particular, linolic acid, linolenic acid, omega-3 fatty acid and omega-6 fatty acid;

- Minerals and trace elements (Ca, P, Fe, Mg, Zn, Cr, Se);

- Pigments (chlorophyll, carotinoides) and phytoenzymes;

-Vitamins and biocatalysts (E, C, B complexes),

these extracts have an excellent health-beneficial and wound-tissue regenerating effect. The minerals, trace elements, enzymes, fatty acids as well as vitamins enhance the metabolism and blood flow and have thus an important and accelerating effect on wound healing by shortening the course of the disease and requiring only minimal after treatment of the wound. Especially important are the unsaturated fatty acids because of their enhancing function as anti-inflammatory agents and accelerators of wound healing. The polysaccharides activate (also locally) the immunoglobulin synthesis and interleukin synthesis as well as the immunoglobulin and interleukin effects.

5. Oxytetracycline (OTC)

OTC is added in quantities of 2 percent to the salve and serves as a well-known broadband antibiotic, in particular, against microbial, bacterial wound infections, primarily against gram-positive bacteria as well as against fungi and yeasts in the wound area.

In the following, the invention will be explained in more detailed based on two basic compositions.

Example 1 (All quantities given in parts by weight).

- specific polyclonal IgY egg yolk antibody of SPF hen 5.0

- oxytetracycline 2.0

- calendula flower extract (fluid extract of calendula 1:2/35 %) 4.0

- microalgae extracts, preferably microalgae extracts of Spirulina platensis in the form of aqueous, alcohol-based (ethanol) or CO2 extracts 2.5

- paraffin oil 25.0

- lanolin (paraffin oil 15.0; water 20.0, cera lanae) ad 100.0

Example 2 (All quantities given in parts by weight).

- specific polyclonal IgY egg yoke antibody of SPF hen 6.0

- oxytetracycline 0.0

- calendula flower extract (fluid extract of calendula 1:2/35 %) 4.0

- microalgae extracts, preferably microalgae extracts of Spirulina platensis in the form of aqueous, alcohol-based (ethanol) or CO₂ extracts 2.5

- paraffin oil 25.00

- lanolin (paraffin oil 15.0; water 20.0, cera lanae) ad 100.00

In the two examples in addition to the mentioned ingredients the following compounds are contained

- as an antioxidant, α-tocopherol (vitamin E) 0.065 %

- as a preservative, ascorbic acid (vitamin C) 0.055 %

The healing and wound-treatment salve described in the examples above can be applied at sub-zero temperatures (degrees centigrade) because it easily becomes liquid on the skin or mucous membrane.

The field of application of the pharmacological and cosmetic products made with the inventive composition, as described in examples 1 and 2, extends to:

- resistant suppurate, inflamed, abscessed, gangrenous-necrotic skin wounds and mucous membrane wounds, as well as bacterial wound infections such as Ulcus cruris or surgery cuts that heal slowly;

- prevention of secondary wound infection in the case of burns, scald injuries, and frostbite of the skin and of the externally accessible mucous membranes of the ear, the oral cavity, genital and anal areas of humans and animals,

- treatment of furuncles as well as discharging skin eczema of humans and animals;

- wound healing of decubitus;

- faster healing and antimicrobial prevention on wounds of all kinds as well as inflammatory changes of the skin and cutaneous mucous membrane as well as penis necrosis and cloacal infections of waterfowl.

In current very broad experimental uses of the composition, side effects in patients have not been observed.

When storing the composition according to the invention such that it is protected against light exposure and is kept at temperatures of +4 degrees Celsius to +8 degrees Celsius, the composition has a shelf life of at least twelve months from the manufacturing date.

The composition according to the invention provides a comprehensive combinatory spectrum of antiphlogistic, regenerative antibiotic and immunizing effects.While specific embodiments of the invention have been shown and described in detail to illustrate the inventive principles, it will be understood that the invention may be embodied otherwise without departing from such principles.

Claims

1. WHAT IS CLAIMED IS:
1.A composition for pharmaceutical products and cosmetic products, the composition comprising:
IgY egg yolk antibodies of SPF (specific pathogen free) hens; and
microalgae extracts selected from the group consisting of Spirulina species and Spirulina varieties and Chlorella species.

2.The composition according to claim 1, further comprising oxytetracycline.

3.The composition according to claim 2, containing 1.1 to 2.5 parts by weight oxytetracycline.

4. The composition according to claim 1, wherein the IgY egg yolk antibodies are monovalent or polyvalent.

5.The composition according to claim 1, containing 4.0 to 10.0 parts by weight of the IgY egg yolk antibodies.

6.The composition according to claim 1, containing 2.0 to 5.0 parts by weight of microalgae extracts.

7.The composition according to claim 1, wherein the Spirulina species and Spirulina varieties are present in the form of aqueous extracts, alcohol-based extracts, or CO₂ extracts.

8.The composition according to claim 1, containing the Chlorella species in the form of aqueous extracts, alcohol-based extracts, or CO₂ extracts.

9.The composition according to claim 1, wherein the microalgae extracts are Spirulina platensis extracts.

10. The composition according to claim 1, further comprising α- tocopherol as an antioxidant.

11.The composition according to claim 1, further comprising ascorbic acid as a natural preservative.

12.A method of treating infected wounds of the skin and mucous membranes, the method comprising the steps of:
preparing a composition comprising IgY egg yolk antibodies of SPF hens and microalgae extracts selected from the group consisting of Spirulina species and Spirulina varieties and Chlorella species;
administering the composition topically onto the infected wound.

13.The method according to claim 12, further comprising the step of preparing a salve from the composition.

14.A method of preventing infection of wounds of skin and mucous membranes, the method comprising the steps of:
preparing a composition comprising IgY egg yolk antibodies of SPF hens and microalgae extracts selected from the group consisting of Spirulina species and Spirulina varieties and Chlorella species;
administering the composition topically onto the wound.

15.The method according to claim 14, further comprising the step of preparing a salve from the composition.
